(19) United States
(12) Patent Application Publication
(10) Pub. No.: US 2015/0099817 A1
(43) Pub. Date: Apr. 9, 2015

(54) MOULDED PARTS MADE OF PMMA POWDER AS SIMPLE DOSING AID IN THE MANUFACTURE OF DENTAL PROSTHESES

(71) Applicant: HERAEUS KULZER GMBH, Hanau (DE)

(72) Inventors: Klaus RUPPERT, Maintal (DE); Kevin KERSCHER, Neu-Anspach (DE); Stephan DEKERT, Neu-Anspach (DE)

(73) Assignee: HERAEUS KULZER GMBH, Hanau (DE)

(21) Appl. No.: 14/299,362

(22) Filed: Jun. 9, 2014

(30) Foreign Application Priority Data
Oct. 6, 2013 (DE) 10 2013 106 018.8

(51) Int. Cl. A61K 6/083; B29C 69/02; A61L 24/06

(52) U.S. Cl. CPC A61K 6/083; A61L 24/06; B29C 69/02; B29L 2031/7722
USPC 521/149; 526/329.7; 525/309; 522/120; 264/109

(57) ABSTRACT

The invention relates to three-dimensional dimensionally-stable moulded parts made of polymeric particles whose particles are connected firmly to each other, at least in part, at the contact sites of the particles, whereby the particles are preferably glued to each other, at least in part, at the contact sites. The pre-packaged moulded parts made of polymeric, powdery particles made of PMMA can take up acrylate or MMA into the porous three-dimensional structure and form a pasty or creamy dough that can be cast, pressed or injected and can be cured by common techniques after the forming. Also subject matters of the invention are the use of and a kit for producing said pasty dough. Said pre-packaged moulded parts are used in methods for producing prostheses, such as dental prostheses, bone cements, investment compounds, in particular of porous substrates, as investment compound in metallography.

The invention relates to three-dimensional dimensionally-stable molded parts made of polymeric particles whose particles are connected firmly to each other, at least in part, at the contact sites of the particles, whereby the particles are preferably glued to each other, at least in part, at the contact sites. The pre-packaged molded parts made of polymeric, powdery particles made of PMMA can take up acrylate or MMA into the porous three-dimensional structure and form a pasty or creamy dough that can be cast, pressed or injected and can be cured by common techniques after the forming. Also subject matters of the invention are the use of and a kit for producing said pasty dough. Said pre-packaged molded parts are used in methods for producing prostheses, such as dental prostheses, bone cements, investment compounds, in particular of porous substrates, as investment compound in metallography.

### BACKGROUND OF THE INVENTION

Prostheses in the field of medical technology, such as dental prostheses or bone prostheses, are manufactured by mixing a powder, which consists essentially of PMMA (poly(methyl-2-methylpropenoate), and a liquid, consisting mainly of MMA. Despite specific instructions of the manufacturers in the instructions for use concerning the mixing ratio of powder and liquid, the majority of users fails to comply exactly with the dosing instructions, but rather doses “by feel” based on the viscosity of the resulting mixture.

The ensuing variations in the materials properties, color appearance and shrinkage behavior of the prostheses thus made are a major problem in this context. Another problem is the content of residual monomer resulting from the non-stoichiometric conversion, when the user fails to comply with the dosing instructions. The increased health hazard experienced by the patients due to these monomers is directly related to the residual monomer content. Moreover, the afore-mentioned shrinkage can lead to inaccuracies of fit in the work produced by the dental laboratory.

It was therefore an object of the invention to devise a method enabling easy and exact dosing of PMMA powder or similar polymeric powders without additional equipment. Another object of the invention was to design the dosing and a method for producing PMMA prostheses, in particular dental prostheses, as well as for investment compounds and other applications, more economical. It was also an object not to impair the mixing and the mixing times with the monomers during the processing and, to the extent possible, to dispense with the use of additional packaging units through customary packing. Moreover, the method should best be automated and minimize the procedural steps carried out at user premises.

Said objects were solved by the molded parts according to claims 1 to 15, which can basically be provided as pre-packaged intermediate products of a three-dimensional shape and essentially have the same processing properties as the powdery PMMA materials. The objects are also solved by the inventive method according to claim 8 and the kit according to claim 19 as well as the use according to claims 16 and 17. Advantageous refinements of the invention are presented in the sub-claims and, in detail, the description. According to the invention, the desired amount of powder is mixed with a defined amount of solvent mixture.

### SUMMARY OF THE INVENTION

The subject matter of the invention are three-dimensional molded parts made of polymeric particles, whereby the polymeric particles comprise powdery particles, powders, grains, pellets, granulates, spherical particles, extrudates, in particular rod-shaped or of any shape known to a person skilled in the art, and/or mixtures of different particles, and the particles are firmly connected to each other, at least in part, at the contact sites of the polymeric particles, in particular are glued to each other, and the polymeric particles consist of organic polymers. Preferably, the molded parts consist of said polymeric particles. In an alternative, the molded parts of the polymeric particles and at least one monomer comprise the ones specified below. Mixtures of different particles can, in particular, also be particles of identical or different geometry. According to an alternative, it is preferred that particles of identical geometry, but different particle size are present in the molded parts. Spherical particles having a defined or various diameters are preferred polymeric particles. Spherical particles are essentially round. The particles can just as well be bead-shaped. Particularly preferred polymeric spherical particles have an aspect ratio of 1.8 or less, in particular of 1.5 or less, preferably of 1.25 or less, particularly preferably of 1.1 or less. The particle sizes are preferably in the range of 1 to 150 μm, preferably different fractions of particle sizes are mixed with each other. Preferably, particles of a fraction of 1 to 50 μm, in particular of 1 to 30 μm, and a second fraction of 75 to 150 μm can be processed into a molded part. It is just as well to use particles of more particle size fractions.

### DETAILED DESCRIPTION

### BRIEF DESCRIPTION OF THE DRAWINGS

FIG. 1 is an SEM image of the molded parts according to the invention, at a resolution of 1 mm,

FIG. 2 is an SEM image of the molded parts according to the invention, at a resolution of 200 micrometers, μm,

FIG. 3 is an SEM image of the molded parts according to the invention, at a resolution of 100 micrometers,

FIG. 4 is an SEM image of a molded part with small and large spherical bodies, at a resolution of 10 micrometers,

FIG. 5 is an SEM image of the molded parts according to the invention, at a resolution of 10 micrometers.

The three-dimensional molded parts obtainable through treating the polymeric, in particular powdery, particles with a solvent, a solvent mixture, optionally together with a monomer, such that only the surfaces of the particles are wetted and the organic particles are partially dissolved or wetted only on the surface. This can be evident from slight swelling of the polymeric particles, preferably polymer beads, at the later contact sites. Therefore, a defined ratio of polymeric particles and solvents, optionally together with monomers, at a defined particle size is to be selected in order to prevent the particles from dissolving too extensively. The weight ratio of polymeric particles, in particular of the spherical particles or powder, and solvent optionally comprising monomers is in the range of approx. 100 to less than 20, in particular 100:15. The swelling time usually is 1 to 2 minutes.

The amount of solvent, and optionally of monomer, is to be selected appropriately such that the polymer beads swell no more than slightly at the contact sites of the individual beads and get glued to each other, while the porosity of the powder and of the resulting molded part remain essentially unaffected.

The invention is based on the core fact that highly porous molded parts are formed whose porosity corresponds to that of the bulk and non-compacted polymeric particles in order not to significantly change the processing properties for the user as compared to the powders. The PMMA particles are often present in the form of beads of a defined diameter.

In general, fractions of particles of different diameters can be used for manufacture of the molded parts. One advantage of the use of different fractions of particles is that it provides the ability to set the processing time for the later conversion with the monomer for producing the doughs for production of the prostheses.

Mixtures of water, a low amount of a short-chain alcohol, such as, e.g. ethanol or methanol, or a ketone, such as, e.g., acetone or MMA, have proven to be particularly useful for producing the molded parts. Aqueous mixtures of alcohols or ketones are therefore used preferably during the production of the molded parts.

According to the invention, the polymeric particles, preferably a PMMA powder, are dispersed in a solvent, solvent mixture optionally containing a monomer, and cast, or optionally pressed, into a mold.

After some time of exposure of the solvent to the polymeric particles, preferably polymer powder, the solvent is then removed slowly at room temperature through vaporizing or evaporation, preferably at slightly elevated temperature (e.g. 50° C.) and/or in a vacuum.

The resulting molded part is sufficiently stable such that it withstands the further production and handling steps, such as detachment from the mold, packaging, etc., without suffering mechanical damage.

The molded part thus produced has a defined mechanical stability. Moreover, the molded part is porous. The porosity of the molded part is adapted to the later processing with monomers, which can be taken up by, preferably absorbed into, the porous molded part. Accordingly, a porous, three-dimensional molded part that takes up at least one monomer, preferably MMA, in a short period of time and disintegrates into a pasty or creamy dough that can be cast, pressed or injected is a subject matter of the invention.

The porous molded part according to the invention can take up, in particular absorb, a liquid whose main component is (meth)methacrylate. Advantageously, the liquid contains more than 80% by weight MMA, in particular 90% by weight MMA or more and, optionally, cross-linkers, initiators, accelerators, activators and/or other auxiliary substances and/or agents.

Subsequently, the molded part disintegrates, at least in part, into the polymeric particles or can be processed after brief mixing, for example by stirring for, in particular, approx. 30 seconds, into a polymeric dough that can be cast, pressed or injected.

It thus becomes feasible to provide the polymeric particles for later processing in pre-packaged dosages in order to process these into a prostheses material with a defined amount of MMA (liquid) in a short time, preferably within approx. 1 to 5 min, advantageously within 30 seconds, into a dough that can be cast, pressed or injected. According to the invention, it is therefore preferred to provide a molded part together with a defined amount of monomer in a kit. Preferably, the needed amount of monomer is provided by means of a bottle dispenser that is screwed onto the glass bottle. Particularly preferably, the kit comprises a large number of pre-packaged molded parts and the corresponding defined amounts of monomer. The monomer can be present in the kit individually packaged in vials or in a cartridge that dispenses a defined amount.

It is a particular advantage of the molded parts according to the invention that their processing and polymerization properties do not differ significantly from those of the bulk polymeric particles, in particular the bulk powders. The properties of the dental products thus produced are significantly more reproducible, since the same mixing ratio is set for each prosthesis, since the material relationships are always the same.

Dimensioning the molded parts appropriately allows defined amounts of polymeric particles to be provided in the form of a defined “intermediate product”. The molded parts can have any conceivable geometry with essentially the shape of a cuboid, cube, rod, cylinder, strand, sphere, truncated cone, disc, ring, waffle-shape, egg-shape (round convex shape), tetrahedron or polyhedron being preferred. One or many molded part(s) can be packaged in packaging means, such as bag or in a box. The molded parts according to the invention can all have identical or different geometry. Preferably, the molded parts have essentially the same mass.

Appropriate dimensioning of the molded part or preforms allows defined amounts of powder to be dosed either through a molded part in the form of a block of the corresponding size, for example 30 g for a dental full prosthesis, or multiple blocks of equal size, for example 3 blocks of 10 g each for a dental full prosthesis, or multiple blocks of different size, for example 1 block of 10 g and 1 block of 5 g for a partial dental prosthesis. Accordingly, providing molded parts of different weights, such as 1 g, 2.5 g, 5 g, 10 g, 15 g, 20 g, etc., all desired amounts of polymeric particles can be combined much like in a modular system. The amounts of monomer are preferably pre-packaged appropriately in the kit or are provided by means of a corresponding dosing device. It is feasible just as well to produce cylindrical molded parts rather than blocks.

According to the invention, the polymeric particles are made of organic polymers comprising acrylate and/or methacrylate polymer.

According to the invention, the polymeric particles are present as acrylate or methacrylate powder in a geometrically defined and stable form as a molded part and are used for producing prosthetic materials.

Also a subject matter of the invention is a three-dimensional molded part made of polymeric particles, in particular spherical particles, whereby the density of the molded parts and the density of the loose bulk particles are approximately similar or preferably almost identical. Accordingly, the bulk density can be 1.1997 g/cm³ and the density of the molded part made from this bulk material can be 1.1978 g/cm³. The molded parts according to the invention therefore preferably have a density of 1.5 g/cm³ or less, in particular 1.4 g/cm³ or less, 1.3 g/cm³ or less, particularly preferably of 1.2 g/cm³ or less. Advantageously, the porosity of the bulk material is essentially maintained in the solid body. The polymeric particles comprise powdery particles, powders, grains, spherical particles, in particular of an aspect ratio of 1.4 or less, pellets, granulates, extrudates and/or mixtures of different particles, and the particles in the molded parts are firmly connected to each other, at least in part, at the contact sites of the polymeric particles, in particular are glued to each other, and the polymeric particles comprise organic polymers such as acrylate and/or methacrylate polymer. The scope of the invention includes, in particular, molded parts of a porosity larger than or equal to 95%, 90%, 85%, 80%, 75% or 70% of the porosity of the bulk polymeric particles of the same shape and particle size. The porosity according to the invention can be determined indirectly by means of the corresponding specific surface of the bulk particles and molded parts.

The scope of the invention also includes molded parts whose specific surface is 70% or more, in particular 75% or more, preferably 80% or more, more preferably 85% or more, particularly preferably 90% or more or 95% or more of the specific surface of the polymeric particles, in particular of the free-flowing bulk polymeric particles from which the molded part was made, i.e. of the polymeric particles that are not connected to each other at the contact sites.

The invention discloses molded parts that are porous and are of appropriate porosity such that the molded parts take up, in particular absorb, (meth)methacrylate rapidly. Moreover, the molded parts disintegrate, at least in part, preferably completely, into the polymeric particles or into a polymeric dough that can be cast, pressed or injected and can be processed for a certain period of time. Preferably, the molded part can be processed into a dough that can be cast, pressed or injected for 2 minutes after being exposed to the monomer.

Also a subject matter of the invention is a molded part of a geometrically defined three-dimensional shape, which is, in particular, dimensionally-stable to a pressure load (pressure resistance of 1 MPa or more, preferably of 1.5 MPa or more, more preferably of 2.0 MPa or more, particularly preferably of 2.25 MPa or more. Advantageously up to 2.45 MPa or more than 2.45 MPa. Additionally or alternatively, the molded parts comprise a diametric tensile strength of 0.1 MPa or more, in particular of 0.2 MPa or more, preferably of 0.3 MPa or more, particularly preferably of 0.4 MPa or more, of 0.44 MPa or more. The pressure resistance of the molded parts made according to the invention was 2.45 MPa. Their diametric tensile strength was 0.44 MPa. The pressure resistance is determined according to DIN EN ISO 9917-1 (Appendix D) and the diametric tensile strength is determined according to ADA specification no. 27.

Molded parts according to the invention comprise polymeric particles comprising polymers, such as homo- and/or co-polymers based on at least one of the monomers, comprising a (meth-)acrylate group selected from methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetrahydrofuryl-acrylate, polypropylene-glycol-mono-acrylate, hydroxyethylacrylate, hydroxypropylacrylate, hydroxyethylmethacrylate, hydroxypropylmethacrylate, a mixture containing at least one of these (meth-)acrylates and/or co-polymers comprising one or at least two of the afore-mentioned monomers. Moreover, the polymers can also comprise mixtures of dental monomers, such as, e.g., MMA and at least one cross-linker. Typical cross-linkers are BDMA, 1,4-butanediol-dimethacrylate (1,4-BDMA) or pentaerythritol-tetraacrylate, urethanedimethacrylate (UDMA), bis-GMA monomer (bisphenol-A-glycidyl-methacrylat). The use of thinners (low viscosity acrylates such as triethyleneglycoldimethacrylate (TEGDMA) and diethyleneglycoldimethacrylate (DEGMA), etc. Further cross-linkers are disclosed in the following also under the polymeric particles comprising co-polymers comprising at least one (meth)acrylate monomer with two, three, four, five or six (meth-)acrylate groups.

The scope of the invention also includes polymeric particles based on at least one (meth-)acrylate monomer with just one (meth-)acrylate group or based on a mixture of at least two of said (meth-)acrylate monomers.

Particularly preferred molded parts according to the invention are characterised in that the molded part contains monomers at the contact sites, at which the polymeric particles are connected to each other, at least in part, in particular they are glued together at the contact sites by monomers, and in that the monomers comprise (meth-)acrylate with a (meth-)acrylate group selected from acrylate, methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetrahydrofuryl-acrylate, polypropylene-glycol-mono-acrylate, hydroxyethylacrylate, hydroxypropylacrylate, hydroxyethylmethacrylate, hydroxypropylmethacrylate and/or a mixture containing at least one of said (meth-)acrylates.

For producing the bone cements, investment compounds, etc., the molded parts are exposed to a curable monomer, such as acrylate or methacrylate or mixture thereof, preferably soaked and processed accordingly, preferably introduced into a mold. Then, the curing can proceed. The curing can proceed by means of self-curing, radiation curing, in particular photo-curing, and/or thermal curing. For example through irradiation with UV light and/or thermally by heating. Common photoinitiators, activators, stabilizers, hot-curing initiators and other common additives or auxiliary agents can be provided together with the monomers or separately.

Another embodiment of the invention relates to a method for producing the molded part and to a molded part obtainable according to said method, comprising
(i). treating polymeric particles, in particular polymeric particles of organic polymers;
(ii). with a solvent or solvent mixture, each optionally comprising at least one monomer to obtain a mixture;
(iii). forming the mixture;
(iv). removing the solvent or solvent mixture;
(v). obtaining the molded part.

It is according to the invention to use in (ii). a monomer, in which the polymeric particles are soluble, preferably with a solvent or solvent mixture, in which the polymeric particles are soluble also and which is/are volatile. Alternatively, it is also preferred according to the invention to use a mixture of alcohol/water or of ketone/water. Preferably, at least one short-chain alcohol having 1 to 4 C atoms, such as methanol, ethanol, or a ketone, such as acetone, an aqueous mixture of one afore-mentioned alcohol or ketone, at least one monomer comprising a (meth-)acrylate having one (meth-)acrylate group or a mixture of the monomer and at least one alcohol or ketone are used as solvent or solvent mixture in (ii), each optionally comprising at least one monomer.

For gluing or partial dissolution of the polymeric particles, a monomer can preferably be used in (ii), which comprises a (meth-)acrylate with one (meth-)acrylate group and is selected from acrylate, methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetra hydrofuryl-acrylate, polypropylene-glycol-mono-acrylate, hydroxyethylacrylate, hydroxypropylacrylate, hydroxyethylmethacrylate, hydroxypropylmethacrylate and/or a mixture containing at least one of said (meth-)acrylates.

According to a preferred variant, the method uses as (ii) solvent or solvent mixture each optionally comprising at least one monomer: a) at least one alcohol comprising methanol and/or ethanol;

b) at least one ketone comprising acetone; c) at least one monomer comprising a (meth-)acrylate with one (meth-)acrylate group, optionally mixed with at least one alcohol, or at least one monomer comprising one (meth-)acrylate with one (meth-)acrylate group, optionally mixed with at least one ketone, preferably a monomer such as MMA mixed with at least one ketone, preferably acetone, or d) aqueous mixture of methanol or ethanol. According to the invention, MMA is used mixed with at least one alcohol, preferably ethanol.

Preferably, the solvent or solvent mixture, each optionally comprising at least one monomer, is used at a weight ratio of 1:200 to 50 to 100, in particular 2:100 to 15:100. A particularly preferred mixture, which can be used to obtain very stable molded parts, comprises 4 to 10 parts by weight of a mixture of ethanol and monomer, such as MMA, and approx. 100 parts by weight polymeric particles.

Also preferably, the mixture of solvent and monomer comprises 1 to 30 parts by weight solvent to 1 to 5 parts by weight monomer, in particular 5 to 20 parts by weight solvent to 1 to 5 parts by weight monomer. According to the invention, approx. 14 parts by weight solvent are used per one part of monomer.

For producing the molded parts, steps (ii) and/or (iii) of the method are carried out in that the solvent or solvent mixture wets the polymeric particles on the surface and, optionally, partially dissolves the polymers. After wetting or partial dissolution, the solvent or solvent mixture is vaporized and the molded part is thus dried, upon which it attains its stability.

The mixture obtainable according to the inventive method is transferred into a mold in step (iii) for forming the molded part, in particular the green compact is formed from which the molded part is formed by means of drying. According to the invention, the mixture is transferred in (iii). into a mold, in particular the mold is a negative image of a cuboid, cube, rod, cylinder, strand, bead, egg-shape (a round convex shape), tetrahedron or polyhedron, preferably the mold is open or can be opened on one side in each case. For formation of the molded part, the afore-mentioned molds can be provided to be a single, two or more parts. In as far as required, the mixture can be pressed in said mold at a defined pressure in order to press together the contact sites of the polymeric sufficiently in order to ensure sufficient connection between the contact sites during the subsequent drying.

For drying, the solvent or solvent mixture is removed in (iv)., preferably through vaporizing, supplying heat, optionally in a vacuum. Particularly well-suited is drying or vaporization of the solvents at temperatures between 20 to 60° C., preferably approx. 50° C., optionally in a vacuum.

Also a subject matter of the invention is the use of the molded parts for reproducible dosing of the polymeric particles. The molded parts according to the invention allow for reproducible and very accurate dosing for a multitude of applications without separate packaging being required. Preferably, the molded parts are used for dosing the polymeric particles in the manufacture of dental prostheses, investment compounds in histology, metallography, veterinary medicine.

Also a subject matter of the invention is the use of the molded parts together with at least one monomer comprising (meth-)acrylate with one (meth-)acrylate group comprising acrylate, methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetrahydrofuryl-acrylate, polypropylene-glycol-mono-acrylate, hydroxyethylacrylate, hydroxypropylacrylate, hydroxyethylmethacrylate, hydroxypropylmethacrylate and/or a mixture containing at least one of said (meth-)acrylates for the manufacture of a pasty or creamy polymeric dough that can be cast, pressed or injected and can be cured by radiation and/or heat or is self-curing.

It is particularly preferable to use the molded parts for dosing the polymeric particles during the production of prostheses, dental prostheses, prosthetic materials, investment compounds in histology, metallography, bone cements, prosthesis in veterinary medicine, investment compound for a porous substrate, in metallography for preparing the micro-structure of a substrate, for embedding transparent sections in materials testing, as investment compound for testing of printed circuit boards, as investment compound for testing of electronic components, as investment compound for testing in semi-conductor technology, as investment compound for testing of micro-electronic components, as investment compound for testing in optical electronics, as investment compound for testing in medical equipment engineering and/or as investment compound for testing of medical instruments, as investment compound or material, in materials testing of a substrate.

According to another embodiment, a subject matter of the invention is a kit comprising at least one molded part (a), in particular a multitude of molded parts, and, separately, at least one defined amount of at least one pre-packaged polymerizable monomer (b), whereby

(a) the at least one molded part according to the invention is made of

(a1) at least one organic polymer that is soluble in (b); and comprises (b) at least one defined amount of a monomer for radical polymerization, comprising (meth-)acrylate with one (meth-)acrylate group, in particular selected from acrylate, methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetrahydrofuryl-acrylate, polypropylene-glycol-mono-acrylate, hydroxyethylacrylate, hydroxypropylacrylate, hydroxyethylmethacrylate, hydroxypropylmethacrylate and/or a mixture containing at least one of said (meth-)acrylates, the kit optionally comprises (c) photoinitiator and, if applicable, a content of activator and, optionally, (d) at least one photoinitiator and/or at least one hot- or self-curing initiator.

Monomer (b) in the kit serves for producing the doughs, such as bone cements or dental prostheses. Preferably, the monomer is divided or dividable into defined portions in order to produce reproducible mixtures of (a) and (b).

Likewise, the polymeric particles can comprise co-polymers with at least two different (meth-)acrylate groups, whereby a co-monomer is based on at least one (meth-)acrylate monomer having two, three, four, five (meth-)acrylate groups and/or six (meth-)acrylate groups or a mixture of at least two of said (meth-)acrylates.

Hereinafter are specified suitable co-monomers that can be used in co-polymers and/or as monomer or co-monomer in (ii) and, optionally, in the later processing of the molded parts into prostheses or investment compounds: (meth-)acrylate having at least two (meth-)acrylate groups selected from ethanedioldimethacrylate, tetraethyleneglycoldimethacrylate, diethyleneglycoldimethacrylate, ethyleneglycoldimethacrylate, polyethyleneglycoldimethacrylate (400) or (600), butanedioldimethacrylate, hexandioldimethacrylate, decanedioldimethacrylate, dodecanedioldimethacrylate, 1,3-butyleneglycoldimethacrylate, dipropyleneglycolmethacrylate, bisphenol-A-dimethacrylate, bisphenol-A-dimethacrylate derivative, such as ethoxylated 2-bisphenol-A-dimethacrylate, trimethylolpropanetrimethacrylate, triethyleneglycoldimethacrylate, 2,2-bis-4-(3-methacryloxy-2-hydroxy-propoxy)-phenylpropane (Bis-GMA), tricyclodecane-dimethanoldimethacrylate, an urethanemethacrylate having at least two methacrylate groups, ethanedioldiacrylate, tetraethyleneglycoldiacrylate, diethyleneglycoldiacrylate, ethyleneglycoldiacrylate, polyethyleneglycoldiacrylate (400) or (600), butanedioldiacrylate, hexanedioldiacrylate, decanedioldiacrylate, dodecanedioldiacrylate, 1,3-butyleneglycoldiacrylate, dipropyleneglycolacrylate, bisphenol-A-diacrylate, bisphenol-A-diacrylate derivative, such as ethoxylated 2-bisphenol-A-diacrylate, trimethylolpropanetriacrylate, triethyleneglycoldiacrylate, 2,2-bis-4-(3-methacryloxy-2-hydroxy-propoxy)-phenylpropane (Bis-GMA), tricyclodecanedimethanoldiacrylate and/or an urethaneacrylate having at least two acrylate groups or a mixture containing at least one of said (meth-)acrylates.

Further suitable co-monomers that can be used in co-polymers and/or as monomer or co-monomer in (ii) and, optionally, in the later processing of the molded parts into prostheses or investment compounds include: (meth-)acrylate with three to six (meth-)acrylate groups selected from (i) with three (meth-)acrylate groups from ethoxylated (15)-trimethylolpropane-triacrylate, ethoxylated 5-pentaerythritoltriacrylate, propoxylated (5.5)-glyceryltriacrylate, trimethylolpropanetrimethacrylate, tris(2-hydroxyethyl)-isocyanurate-triacrylate, and/or (ii) with four (meth-)acrylate groups from di-trimethylolpropane-tetra-acrylate, ethoxylated (4)-pentaerythritol-tetra-acrylate, pentaerythritol-tetra-acrylate, di-trimethylolpropane-tetra-methacrylate, ethoxylated (4)-pentaerythritol-tetra-methacrylate, pentaerythritol-tetra-methacrylate and/or (iii) with five (meth-)acrylate groups from di-pentaerythritol-pentaacrylate, i-pentaerythritol-pentamethacrylate, dipentaerythritol-pentaacrylate, di(tetramethylolmethane)-pentamethacrylate and/or (iv) with six (meth-)acrylate groups a dipentaerythritol-hexa(meth)acrylate. Also well-suited are oligomers of (meth-)acrylates, in particular urethane-di-acrylate oligomer.

The invention is illustrated in more detail in the following based on examples, without limiting it to said examples:

### EXAMPLE

Production of the mixture: A mixture was produced from 6.5 ml ethanol and 0.5 ml methylmethacrylate at room temperature.

Production of the dispersion: The mixture described above was added to 100 g PMMA powder (e.g. PalaXpress) at room temperature and mixed intensively through stirring for approx. 2 min.

Production of the molded part: The dispersion was pressed into a mold of the desired geometry. Alternatively, a suspension can be cast into a three-dimensional mold.

The mold typically consists of plastic material and is resistant to the solvents used at the temperatures used. Preferred molds are produced from flexible or elastic plastic materials. Three-dimensional silicone molds have proven to be particularly well-suited, since the part can be removed particularly easily from the mold. In general, multi-part, such as two-part metal molds or multi-part non-flexible molds made of other materials can be used just as well. The molds that can be used are not limited to the examples given, but can generally be used for producing molded parts according to the invention.

The mixture can be brushed into a cylindrical plastic mold. After drying, cylindrical test bodies of approx. 30 g (height 35 mm, diameter 40 mm) can be removed. The pressure resistance of molded parts according to the invention was measured to be 2.45 MPa. The diametric tensile strength was determined to be 0.44 MPa. The pressure resistance is determined according to DIN EN ISO 9917-1 (Appendix D) and the diametric tensile strength is determined according to ADA specification no. 27.

Drying: The solvent mixture is allowed to vaporize slowly either at room temperature or at slightly elevated temperatures (approx. 45° C.). After the solvent is vaporized or nearly vaporized, the molded part attains its mechanical and geometrical stability.

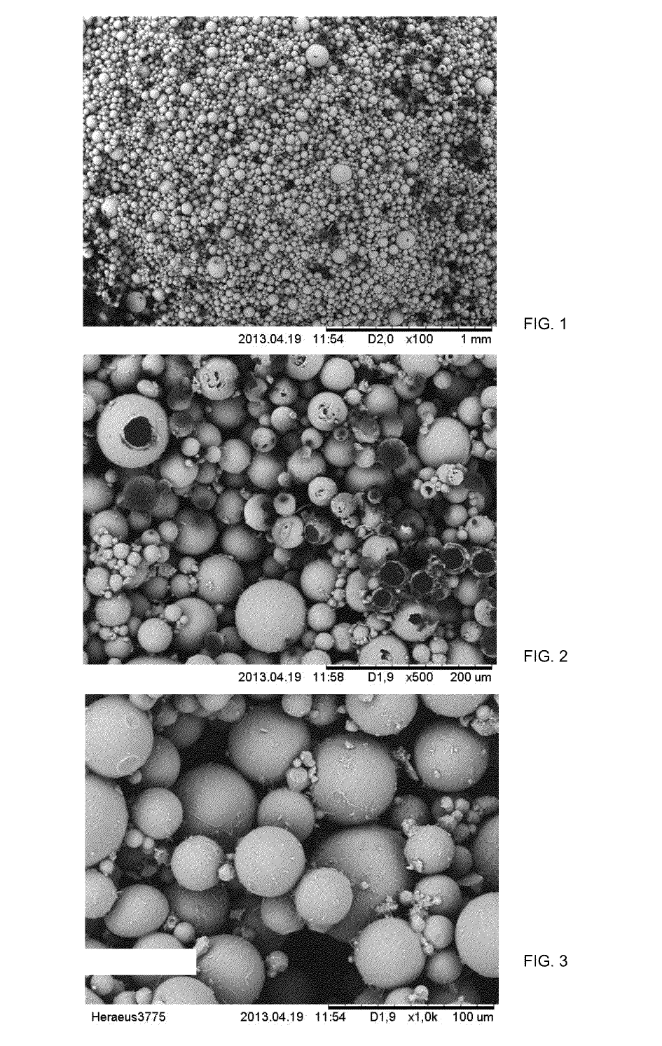

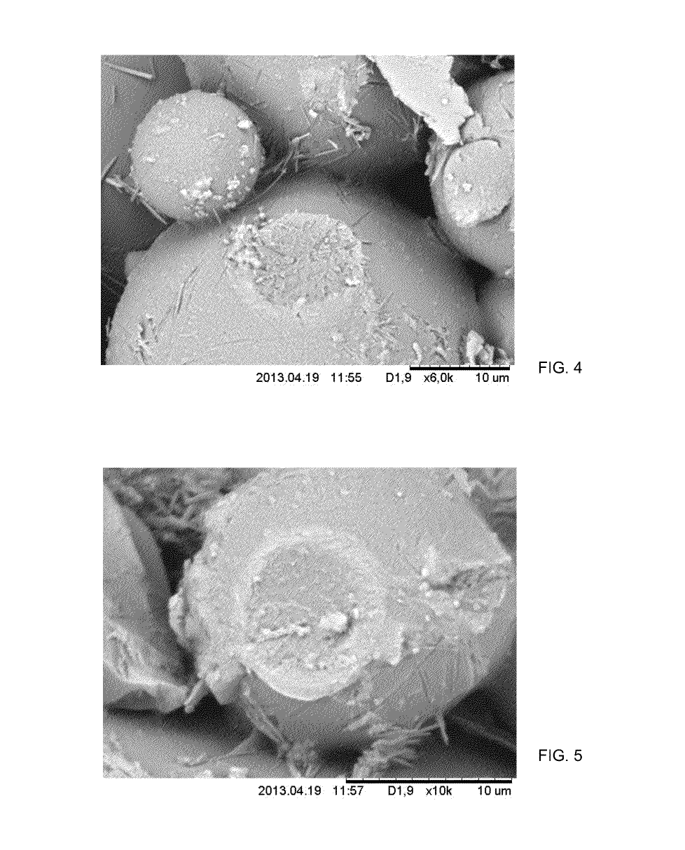

If applicable, the molded part can be re-dried again after removal from the mold by slight heating in a vacuum. FIGS. 1 to 5 show SEM images of the molded parts according to the invention at different resolutions. Resolution FIG. 1 (1 mm), FIG. 2 (200 micrometre, μm), FIG. 3 (100 micrometre), FIG. 4 (10 micrometre), FIG. 5 (10 micrometre). The contact sites of the polymeric particles of the molded parts are seen particularly well in FIGS. 4 and 5 at the level surfaces of the spherical particles (beads). FIG. 4 shows an SEM image of a molded part with small and large spherical particles. The preservation of the porosity in the molded parts can be seen quite well in FIGS. 1 to 5.

1. Three-dimensional molded part made from polymeric particles, wherein the polymeric particles comprise powders, grains, pellets, granulates, spherical particles, extrudates and/or mixtures of different particles, and the particles are firmly connected to each other, at least in part, at the contact sites of the polymeric particles, are glued to each other, and the polymeric particles are formed of organic polymers.

2. Molded part according to claim 1, wherein the organic polymers comprise acrylate and/or methacrylate polymer.

3. Molded part according to claim 1, wherein the molded part is present in a defined three-dimensional geometric shape and, is dimensionally-stable with respect to a pressure load of 1 MPa or more and/or has a diametric tensile strength of 0.1 MPa or more.

4. Molded part according to claim 1, wherein the molded part is porous and the porosity of the molded part is such that the moulded part absorbs a liquid whose main component is (meth)methacrylate, and the moulded part disintegrates, at least in part, into the polymeric particles or can be processed after brief mixing into a polymeric dough that can be cast, pressed or injected.

5. Molded part according to claim 1, wherein the polymeric particles contain polymers based on the monomers, comprising at least one (meth-)acrylate group comprising methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetrahydrofuryl-acrylate, polypropylene-glycol-mono-acrylate, hydroxyethylacrylate, hydroxypropylacrylate, hydroxyethylmethacrylate, hydroxypropylmethacrylate, a mixture containing at least one of these (meth-)acrylates and co-polymers containing at least one or at least two of the afore-mentioned monomers.

6. Molded part according to claim 1, wherein the moulded part contains monomers at the contact sites, at which the polymeric particles are connected to each other, at least in part, being glued together, comprising (meth-)acrylate with one (meth-)acrylate group comprising acrylate, methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetrahydrofuryl-acrylate, polypropylene-glycol-mono-acrylate, hydroxyethylacrylate, hydroxypropylacrylate, hydroxyethylmethacrylate, hydroxypropylmethacrylate and mixtures containing at least one of said (meth-)acrylates.

7. Molded part according to claim 1, wherein the specific surface of the molded part is at least 80% of the specific surface of the polymeric particles from which the molded part was made.

8. Molded part according to claim 1, wherein the molded part essentially takes the shape of a cuboid, cube, rod, cylinder, strand, bead, roundish convex shape, tetrahedron or polyhedron.

9. Method for producing the molded part of claim 1, comprising
(i). treating polymeric particles of organic polymers;
(ii). with a solvent or solvent mixture, each optionally comprising at least one monomer to obtain a mixture;
(iii). forming the mixture;
(iv). removing the solvent or solvent mixture;
(v). obtaining the molded part.

10. Method of claim 9, wherein the solvent or solvent mixture in (ii), each optionally comprising at least one monomer, comprises at least one short-chain alcohol having 1 to 4 C atoms, a ketone, an aqueous mixture of one afore-mentioned alcohol or ketone, optionally at least one monomer comprising a (meth-)acrylate having one (meth-)acrylate group or a mixture of the monomer and at least one alcohol or ketone.

11. Method according to claim 10, wherein the solvent or solvent mixture in (ii), each optionally comprising at least one monomer, comprises
a) at least one alcohol comprising methanol and/or ethanol;
b) at least one ketone comprising acetone;
c) at least one monomer comprising a (meth-)acrylate with one (meth-)acrylate group, optionally mixed with at least one alcohol and/or ketone, or
d) aqueous mixture of methanol or ethanol or
e) aqueous mixture containing acetone.

12. Method according to claim 9, wherein the solvent or solvent mixture in (ii) and/or (iii) wets the polymeric particles on the surface and, optionally, partially dissolves the polymers.

13. Method according to claim 9, wherein the solvent or solvent mixture in (ii). is removed through vaporizing, supplying heat, optionally in a vacuum.

14. Method according to claim 9, wherein the mixture is transferred into a mold in (iii)., whereby the mold takes the shape of a cuboid, cube, rod, cylinder, strand, bead, roundish convex shape, tetrahedron or polyhedron.

15. Molded part obtained by the method of claim 1.

16. Method for reproducible dosing of polymeric particles, which comprises dosing said polymeric particles with the molded part of claim 1.

17. Method for the manufacture of a pasty polymeric dough that can be cast or injected and can be cured by radiation and/or heat, which comprises manufacturing said pasty polymeric dough with the molded part of claim 1 together with at least one monomer comprising (meth-)acrylate with one (meth-)acrylate group comprising acrylate, methylmethacrylate, ethylenemethacrylate, propylmethacrylate, butylmethacrylate, n-hexylmethacrylate, 2-phenoxyethylmethacrylate, isobornylmethacrylate, isodecylmethacrylate, polypropylene-glycol-mono-methacrylate, tetrahydrofuryl-methacrylate, polypropylene-glycol-mono-methacrylate, methylacrylate, ethyleneacrylate, propylacrylate, butylacrylate, n-hexylacrylate, 2-phenoxyethylacrylate, isobornylacrylate, isodecylacrylate, polypropylene-glycol-mono-acrylate, tetrahydrofuryl-acrylate, polypropylene-glycol-mono-acrylate, hydroxyethylacrylate, hydroxypropylacrylate, hydroxyethylmethacrylate, hydroxypropylmethacrylate and/or a mixture containing at least one of said (meth-)acrylates.

18. Method of claim 16 for dosing the polymeric particles during the production of prostheses, dental prostheses, prosthetic materials, investment compounds in histology, metallography, bone cements, prosthesis in veterinary medicine, of spacers, investment compound for a porous substrate, in metallography for preparing the micro-structure of a substrate, for embedding transparent sections in materials testing, as investment compound for testing of printed circuit boards, as investment compound for testing of electronic components, as investment compound for testing in semi-conductor technology, as investment compound for testing of micro-electronic components, as investment compound for testing in optical electronics, as investment compound for testing in medical equipment technology and/or as investment compound in the testing of medical instruments, as investment compound in the materials testing of a substrate.

19. Kit comprising at least one molded part (a) and, separately, at least one defined amount of at least one pre-packaged polymerizable monomer (b), wherein
(a) the at least one part according to claim 1 is made from
(a1) at least one organic polymer that is soluble in (b); and comprises
(b) at least one defined amount of a monomer for radical polymerization, comprising at least one (meth-)acrylate with one (meth-)acrylate group optionally comprising (c) photoinitiator, optionally a content of activator and, optionally,
(d) at least one photoinitiator and/or at least one hot- or self-curing initiator.

20. Molded part according to claim 1, wherein the particles that are firmly connected to each other, at least in part, at the contact sites of the polymeric particles, are glued to each other.
